(19) United States
(12) Patent Application Publication
(10) Pub. No.: US 2006/0039992 A1
(43) Pub. Date: Feb. 23, 2006

(54) Novel compositions and methods utilizing green-lipped mussel and fatty acids

(76) Inventors: Bruce A. Miller JR., Mt. Bethel, PA (US); Olav E. Sandnes, Mt. Bethel, PA (US); John S. Ryan, Takapuna (NZ)

Correspondence Address: PATE PIERCE & BAIRD, 215 SOUTH STATE STREET, SUITE 550, PARKSIDE TOWER, SALT LAKE CITY, UT, 84111, US

(21) Appl. No.: 11/141,995

(22) Filed: Jun. 1, 2005

Related U.S. Application Data
(60) Provisional application No. 60/576,210, filed on Jun. 2, 2004.

(51) Int. Cl. A61K 35/56; A61K 31/202

(52) U.S. Cl. 424/547; 514/560

(57) ABSTRACT

Disclosed herein are compositions containing synergistic combinations of marine mollusk tissue and at least one fatty acid configured into a delivery formulation suitable for administration to humans and animals. A preferred embodiment of the compositions of the present invention may utilize an extract of green-lipped mussel and at least one polyunsaturated fatty acid selected from eicosapentaenoic acid (EPA), docosahexaenoic acid (DHA), eicosatetraenoic acid (ETA), docosapentaenoic acid (DPA), and combinations thereof, and a delivery formulation selected from tablets, capsules, liquids, oils, suspensions, emulsions, solutions, and powders. Also disclosed herein are methods for ameliorating and/or mitigating inflammatory-mediated conditions. A preferred embodiment of the methods of the present invention may include: (1) selecting a marine mollusk extract known to reduce inflammation; (2) selecting at least one fatty acid known to reduce inflammation; (3) incorporating an effective amount of marine mollusk extract and polyunsaturated fatty acid into a suitable delivery formulation; and (4) administering a delivery formulation to humans and animals to ameliorate an inflammation-mediated disorder.

### RELATED APPLICATIONS

This application claims the benefit of U.S. Provisional Patent Application Ser. No. 60/576,210, filed Jun. 2, 2004, and entitled “NOVEL COMPOSITIONS AND METHODS UTILIZING GREEN-LIPPED MUSSEL AND FATTY ACIDS,” which is hereby incorporated herein by reference.

### BACKGROUND

1. Field of the Invention

This invention relates to synergistic compositions, methods of production, and methods to reduce inflammation in humans and animals and ameliorate musculoskeletal and inflammation-mediated illnesses in humans and animals and, more particularly, to compositions utilizing combinations of marine mollusk and fatty acids and, still more particularly, to combinations of green-lipped mussel and polyunsaturated fatty acids, methods of production, and methods of use for arthritis and other inflammatory conditions.

2. The Background Art

Inflammation-mediated disorders are known to significantly contribute to health related morbidity and mortality throughout all human populations in the world. In addition, inflammation-mediated disorders are known to affect the health and quality of life in non-human animals including, for example and not by way of limitation, dogs, cats, horses, cows, pig, goat, and the like.

The underlying cause of inflammation in many of these disorders remains unknown. However, some conditions may be initiated by microorganisms and related toxins, parasites, chemical and/or biochemical damage, physical damage (e.g., trauma, temperature changes, radiation, and the like), immunological reactions (e.g., hypersensitivity reactions, auto-immune disorders, and the like), and genetic abnormalities.

Inflammation-mediated disorders may affect nearly every organ system in the body of humans and animals including, for example and not by way of limitation, the musculoskeletal, cardiovascular, neurological, respiratory, immunological, genito-urinary, endocrine, dermatological, renal, hepatic, and gastro-intestinal systems. The musculoskeletal system is particularly susceptible to inflammatory-mediated disorders and often manifests as a form of arthritis (e.g., osteoarthritis, rheumatoid arthritis). Signs and/or symptoms of arthritis may include, pain, swelling, redness, localized heat, loss of sensation, deformity, loss of function, loss of mobility, loss of strength, or combinations thereof, in at least one joint, bone, muscle, tendon, ligament or combinations thereof.

Those skilled in the art have attempted to provide compositions and/or methods for alleviating the symptoms of inflammation-mediated disorders. Prior art strategies for ameliorating and/or mitigating the effects of inflammation-mediated disorders have attempted to harvest the inflammation blocking effects of corticosteroids, non-steroidal anti-inflammatory drugs (NSAIDs), salicylates, and histamine receptor antagonists. Although these agents may have modest effect in some inflammation-mediated disorders, they have not been successful in ameliorating and/or mitigating inflammation in selected disease conditions, or in the alternative, have any of several untoward effects (i.e., side effects) which limit use in humans and animals. Moreover, many of these agents may be uneconomical or otherwise cost prohibitive for routine use, owing to cost of production and/or cost therapeutic monitoring for safety and/or efficacy. In addition, these agents may not be readily available for use in synergistic combinations with other anti-inflammatory products.

Therefore, what is needed are compositions containing synergistic combinations of anti-inflammatory compounds and methods for producing these compositions. In addition, methods of using synergistic compounds are needed to ameliorate and/or mitigate inflammation-mediated conditions, disorders, and illnesses. Novel compositions and methods providing useful, economical, efficacious and safer alternatives to the prior art are needed. Such novel compositions and methods are disclosed and taught herein.

### BRIEF SUMMARY AND OBJECTS OF THE INVENTION

A primary object of the present invention is to provide novel compositions with synergistic combinations of anti-inflammatory compounds, methods for producing said novel compositions and methods for using said novel compositions to ameliorate and/or mitigate inflammatory-mediated disorders.

It is also an object of the present invention to provide novel compositions with synergistic combinations of marine mollusk and fatty acid compounds and methods for using said compositions to ameliorate and/or mitigate inflammatory-mediated disorders.

It is a further object of the present invention to provide novel compositions with synergistic combinations of marine mollusk and fatty acid compounds, which may utilize ground green-lipped mussel tissue, a concentrated extract of green-lipped mussel known to have beneficial anti-inflammatory effects, and combinations thereof, and which utilize at least one polyunsaturated fatty acid (PUFA).

It is a still further object of the invention to provide novel compositions with synergistic combinations of marine mollusk and fatty acid compounds, which utilize green-lipped mussel extract in a dried and/or powdered form.

In addition, it is an object of the present invention to provide novel compositions with synergistic combinations of marine mollusk and fatty acid compounds, wherein the PUFAs are from fish sources and/or in the form of an oil.

Moreover, it is an object of the present invention to provide novel compositions with synergistic combinations of marine mollusk and fatty acid compounds in which at least one PUFA is selected from eicosapentaenoic acid (EPA), docosahexaenoic acid (DHA), eicosatetraenoic acid (ETA), and docosapentaenoic acid (DPA).

Additionally, it is an object of the present invention to provide novel compositions with synergistic combinations of marine mollusk and fatty acid compounds in which novel compositions are substantially without side effects and therefore have increased safety compared to prior art compositions and methods.

It is also an object of the present invention to provide novel compositions with synergistic combinations of marine mollusk and fatty acid compounds, which use only natural products as ingredients.

Moreover, it is an object of the present invention to provide novel compositions with synergistic combinations of marine mollusk and fatty acid compounds wherein there is a reduced need for therapeutic monitoring of anti-inflammatory agents.

It is a further object of the present invention to provide novel compositions with synergistic combinations of marine mollusk and fatty acid compounds in which there is a gastro-protective effect.

It is a still further object of the present invention to provide novel compositions with synergistic combinations of marine mollusk and fatty acid compounds wherein the degree of anti-inflammatory relief exceeds that which would be expected from the use of the concentrations of either ingredient alone.

It is also an object of the present invention to provide novel compositions with synergistic combinations of marine mollusk and fatty acid compounds wherein the synergistic combinations utilize a powdered form of PUFAs and may be prepared and administered in the form of capsules, tablets or other pharmaceutical formulations known to those skilled in the art.

Also, it is an object of the present invention to provide novel compositions with synergistic combinations of marine mollusk and fatty acid compounds and methods for ameliorating and/or mitigating an inflammation-mediated disorder involving an organ system selected from the musculoskeletal, cardiovascular, neurological, respiratory, immunological, genito-urinary, endocrine, dermatological, renal, hepatic, and gastro-intestinal systems.

It is another object of the present invention to provide novel compositions with synergistic combinations of marine mollusk and fatty acid compounds, which may be used in combination with traditional inflammation modifying agents (e.g., corticosteroids, NSAIDs, salicylates, histamine receptor antagonists) and/or pain-relieving agents (e.g., acetaminophen).

It is a further object of the present invention to provide novel compositions with synergistic combinations of marine mollusk and fatty acid compounds wherein the morbiditiy and/or mortality from inflammation-mediated conditions may be reduced in humans and animals.

Consistent with the foregoing objects, and in accordance with the invention as embodied and broadly described herein, it has been found that novel compositions comprising combinations of marine mollusk and fatty acid compounds are effective in ameliorating and/or mitigating inflammation-mediated conditions, disorders, and illnesses in humans and animals. As appreciated, novel compositions containing combinations of marine mollusk and fatty acids work synergistically to modulate the biochemical mechanisms and pathways responsible for inflammation in humans and animals. Although the components of the novel compositions of the present invention occur in nature (i.e., green-lipped mussel and PUFAs), it is the novel combinations of these compounds as taught by the present invention that have not heretofore been taught or disclosed in the prior art. Accordingly, the present invention contemplates the therapeutic use of novel compositions with synergistic combinations of marine mollusk and fatty acids for ameliorating and/or mitigating inflammation-mediated disorders including, for example and not by way of limitation, arthritis, allergy, generalized aches and pains, combinations thereof, and the like.

One presently preferred embodiment of a method for using the present invention may include, for example and not by way of limitation: (1) selecting an extract of green-lipped mussel known to have beneficial anti-inflammatory effects; (2) selecting a fatty acid from the group of polyunsaturated fatty acids, eicosapentaenoic acid (EPA), docosahexaenoic acid (DHA), eicosatetraenoic acid (ETA), docosapentaenoic acid (DPA), or combinations thereof; (3) incorporating an effective amount of said green-lipped mussel and fatty acid into a suitable delivery formulation; and (4) administering the formulation containing an effective amount of green-lipped mussel and fatty acid to a human or animal experiencing an inflammation-mediated condition.

### DETAILED DESCRIPTIONS OF THE PREFERRED EMBODIMENTS

It will be readily understood that the components of the present invention, as generally described herein, could be arranged and designed in a wide variety of different configurations. Those of ordinary skill in the art will, of course, appreciate that various modifications to the details herein may be made without departing from the essential characteristics of the invention, as described. Thus, the following more detailed description of the embodiments of the compositions and methods of the present invention is not intended to limit the scope of the invention, as claimed, but it is merely representative of the presently preferred embodiments of the invention.

Novel compounds of the present invention may be administered orally in the form of tablets, capsules, liquids, suspensions, emulsions, solutions, or other means suitable for such ingestion, perhaps as an admixture with other compounds to enhance absorption into the blood stream or to otherwise assist in achieving the desired effects. Likewise, oral administration is contemplated herein to include sublingual (i.e., under the tongue) dosage forms. Novel compounds of the present invention may also be delivered by intranasal (i.e., through the nasal structures), transmucosal (i.e., across mucous membranes) or transdermal (i.e., through the skin) administration.

In addition, novel compounds of the present invention may also be administered parenterally, as a subcutaneous, intramuscular or intravenous injection, or by way of an implant for sustained release. When administered parenterally, the compounds of the present invention are to be dissolved in physiologically acceptable liquid media and/or otherwise compounded in accordance with the known pharmaceutical art.

Unless otherwise defined, the technical, scientific and medical terminology used herein has the same meaning as understood by those informed of the art to which this invention belongs. However, for the purposes of establishing support for various terms that are used in the present application, the following technical comments, definitions and review are provided for reference.

“Ameliorate” may refer to a verb meaning to make better or improve a condition. This may be sometimes used synonymously with “mitigate” which may refer to a verb meaning to reduce or make less severe, or reduce the pain or intensity of some condition.

“Autoimmune” nay refer to a condition wherein components of the immune system (e.g., antibodies or T cells) begin to attack the molecules, cells, or tissues of the organism producing them. Autoimmune conditions frequently trigger the immune system to generate an inflammation-mediated attack on a cell, tissue, organ system and combinations thereof.

“Glucocorticoid” may refer to a corticoid that has a primary effect on carbohydrate metabolism (e.g., cortisone, hydrocortisone, prednisone, prednisolone, triamcinolone, methylprednisolone, dexamethasone, betamethasone and the like).

“Inflammation” may refer to an immune system response to cellular injury (as by infection or trauma). Inflammation is a process for removing and/or destroying injured cells and tissues. Inflammation may be characterized at the site of injured tissue by capillary dilatation, accumulation of leukocytic cells (i.e., infiltration) and localized heat. Often a mammal may also associate pain with inflammation. Inflammation is an important mechanism for the body to control foreign agents and eliminate damaged cells and tissues.

Morbidity and mortality associated with inflammation-mediated conditions is significant throughout the world. Inflammation-mediated conditions afflict both human and animals. In addition, these conditions may contribute to staggering economic costs in terms of health care and lost work productivity. Inflammation-mediated may also have an emotional toll on those afflicted relative to a reduction in quality of life.

As appreciated, those skilled in the art have long-recognized that the process of inflammation is both helpful and hindrance to humans and animals. Inflammation may be an important mechanism allowing the body of a human or animal to identify and destroy foreign and/or potentially dangerous particles. Without an effective inflammation mechanism, humans and animals would have significant difficulty in maintaining proper physiological function including, for example and not by way of limitation, mounting a defense to invading microorganisms and exogenous environmental toxins that may be introduced to the body.

At the same time, it may also be desirous to modulate inflammation-mediated responses to reduce the systemic effects of inflammation, such as, swelling, pain, redness, loss of mobility, loss of function, loss of sensation, deformity and the like. The inflammation cascade may often depend upon chemical mediators to serve as signals for the mobilization and accumulation of immune system cells (i.e., infiltration, chemotaxis) nearby an injured cell, tissue, invading microorganism, exogenous environmental toxin, or the like.

Chemical mediators of inflammation may include, for example and not by way of limitation, vasoactive amines (e.g., histamine, 5-hydroxytryptamine), cytokines, lymphokines, eicosanoids, kinins, fibrinopeptides, and complement factors. Eicosanoids have recently received significant evaluation as inflammatory mediators. Eicosanoids may include prostaglandins, thromboxanes, and leukotrienes. Eicosanoids may be produced from free arachidonic acid (AA) which may have been liberated from cell membrane lipids by a phospholipase enzyme. Free AA may be further modified by prostaglandin synthase and cyclooxygenase (COX) to produce prostaglandins and/or thromboxanes. Likewise free AA may be further modified by lipoxygenase to produce leukotrienes. All of these eicosanoids may play important and separate roles in amplifying the effects of inflammation. Under some circumstances, arachidonic acid-derived eicosanoids modulate the production of pro-inflammatory and immunoregulatory cytokines and overproduction of these compounds is associated with chronic inflammatory diseases.

As appreciated, it may be desirous to develop medicines and other therapeutic agents configured to ameliorate and/or mitigate inflammation-mediated conditions. More particularly, modulating the biosynthesis of eicosanoids may be an important target for the development of medicinal and/or therapeutic agents directed to modifying inflammation. Traditional medicinal and therapeutic compounds to modify inflammation may be broadly classified as corticosteroids (e.g., glucocorticoids), non-steroidal anti-inflammatory drugs (NSAIDs), or salicylates. Histamine receptor antagonists may also modify inflammation, but may not directly modify eicosanoid biosynthesis.

Glucocorticoids may inhibit the action of phospholipase enzymes to liberate or otherwise free arachidonic acid from membrane lipids. Lipoxygenase inhibitors, as their name implies, prevent the conversion of free AA into leukotrienes. NSAIDs and salicylates are believed to inhibit the conversion of free AA into prostaglandins and/or thromboxanes.

Recently, the prior art has disclosed that there may be multiple subtypes of enzymes involved in eicosanoid biosynthesis. In particular, there may now be at least two, and probably more, subtypes of COX enzymes. These may be designated as COX-1 and COX-2, respectively. Traditional NSAIDs agents (e.g., ibuprofen, piroxicam, naproxen, phenylbutazone, and the like) and salicylates (e.g., aspirin) are believed to be non-selective relative to their inhibition of COX. This may be a significant factor in the precipitation of untoward effects (i.e., side effects) associated with these agents.

For example, and not by way of limitation, certain eicosanoids are known to help provide protection to the mucosal lining of the gastro-intestinal system. By administering a non-selective COX inhibitor to a human and animal, there may be a reduced level of inflammation and alleviation of some symptoms. However, there may also be an occurrence of gastro-intestinal side effects (e.g., ulcer). The identification of COX subtypes has resulted in the development of a new class of NSAID compounds known as COX-2 inhibitors. COX-2 inhibitors, as their name implies, may be selective for the COX subtype which potentiates inflammation, while minimizing effects on the gastro-intestinal and other organ systems.

In the search for natural alternatives to anti-inflammatory compounds, marine mollusk, and in particular, New Zealand green-lipped mussel (Perna canaliculus), has been identified as possessing COX inhibiting activity. Green-lipped mussel may contain compounds that have COX inhibiting activity consistent with the effects of COX-2 inhibiting agents. Because green-lipped mussel occurs naturally, it may be a more readily available, and an economically reasonable source of COX inhibiting compounds. In addition, green-lipped mussel may provide a superior, if not similar risk to benefit consideration when compared to traditional NSAIDs and the newer COX-2 inhibitors, respectively.

Another strategy for modulating the biosynthesis of eicosanoids may be to substitute n-3 polyunsaturated fatty acids (PUFAs) for saturated fatty acids, which are normally in abundance in the diets of humans and animals. PUFAs may be incorporated into cell membranes or may otherwise function to reduce the ability of phospholipase to liberate arachidonic acid from cell membranes. By reducing the availability of free arachidonic acid, there may be a resulting decrease in the production of prostaglandins, thromboxanes, and leukotrienes. Scientific and clinical investigations have disclosed that humans and/or animals with diets rich in PUFAs are at reduced risks of developing selected inflammation-mediated conditions.

As appreciated, there is no teaching, disclosure or motivation in the prior art which contemplates the novel combinations containing compositions of green-lipped mussel (or extracts thereof) and fatty acids to work in synergy for the amelioration and/or mitigation of inflammation-mediated disorders. The inventors of the present invention have formulated compounds of the present invention and found that this synergistic combination contain green-lipped mussel and fatty acids, and more particularly, green-lipped mussel tissue and PUFAs, have an anti-inflammatory effect that exceeds the relief which would be expected from the use of either ingredient alone.

Additionally, novel compositions and methods of the present invention may utilize at least one pharmaceutical excipient to increase palatability, improve a pharmaceutical property, or both. As appreciated, a pharmaceutical property may include, for example and not by way of limitation, shelf-stability, half-life, pH, preservation, and the like. Likewise, a pharmaceutical excipient may include, for example and not by way of limitation, stabilizers, acidifiers, neutralizers, anti-microbials, preservatives, emulsifiers, suspending agents, lubricants, binders, disintegrants, solvents, sweeteners, de-bitterizing agents, odor masking agents, texture modifiers and the like.

One presently preferred embodiment of the present invention for ameliorating and/or mitigating inflammation-mediated conditions may include a novel composition of green-lipped mussel and at least one PUFA which is delivered to a human and animal in an amount effective to reduce inflammation.

As further contemplated herein, one presently preferred embodiment of the present invention for ameliorating and/or mitigating immune-mediated conditions may include a novel composition of green-lipped mussel tissue extract and a combination of EPA and DHA, which may then be delivered to a human and animal in an amount effective to reduce inflammation.

In addition, one presently preferred embodiment of the present invention for ameliorating and/or mitigating immune-mediated conditions as contemplated herein may include a novel composition of green-lipped mussel tissue extract and ETA and/or DPA, which maybe delivered to a human and animal in an amount effective to reduce inflammation.

A still further presently preferred embodiment of the present invention for ameliorating and/or mitigating inflammation-mediated conditions may include a novel composition of green-lipped mussel, at least one PUFA selected from EPA, ETA, DPA, DHA, and at least one pharmaceutical excipient, which maybe delivered to a human and animal in an amount effective to reduce inflammation.

While past research by those skilled in the art has attempted to isolate, identify, and characterize new compositions and methods to ameliorate and/or mitigate inflammation-mediated conditions, or compositions with improved risk to benefit profiles, the novel compositions and methods of the present invention combining green-lipped mussel with PUFAs have not heretofore been identified or evaluated. The novel compositions of green-lipped mussel and PUFAs and methods for using said compositions to ameliorate and/or mitigate inflammation-mediated conditions in humans and animals as contemplated by the present invention is therefore a significant advancement in the art.

As appreciated, the present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A composition for ameliorating inflammation-mediated conditions in humans and animals, the composition comprising an effective amount of a marine mollusk tissue and at least one fatty acid.

2. The composition as defined in claim 1, wherein inflammation-mediated conditions may involve a disorder of at least one organ system selected from the group consisting of musculoskeletal, cardiovascular, neurological, respiratory, immunological, genito-urinary, endocrine, dermatological, renal, hepatic, and gastro-intestinal systems.

3. The composition as defined in claim 1, wherein an inflammation-mediated condition is selected from the group consisting of osteoarthritis and rheumatoid arthritis.

4. The composition as defined in claim 1, wherein the marine mollusk tissue is green-lipped mussel (Perna canaliculus).

5. The composition as defined in claim 1, wherein the marine mollusk tissue is an extract of green-lipped mussel (Perna canaliculus).

6. The composition as defined in claim 5, wherein the extract of green-lipped mussel is dried.

7. The composition as defined in claim 5, wherein the extract of green-lipped mussel extract is in powder form.

8. The composition as defined in claim 1, wherein a fatty acid is a polyunsaturated fatty acid.

9. The composition as defined in claim 8, wherein the polyunsaturated fatty acid is selected from the group consisting of eicosapentaenoic acid (EPA), docosahexaenoic acid (DHA), eicosatetraenoic acid (ETA), docosapentaenoic acid (DPA), or combinations thereof.

10. The composition as defined in claim 8, wherein the polyunsaturated fatty acid is derived from fish sources.

11. The composition as defined in claim 1, wherein the composition is provided in a delivery formulation selected from the group consisting of tablets, capsules, liquids, oils, suspensions, emulsions, solutions, and powders.

12. A composition for ameliorating inflammation-mediated conditions in humans and animals, the composition comprising:
an extract of green-lipped mussel tissue in an amount effective to reduce eicosanoid biosynthesis;
at least one polyunsaturated fatty acid selected from the group consisting of eicosapentaenoic acid (EPA), docosahexaenoic acid (DHA), eicosatetraenoic acid (ETA), docosapentaenoic acid (DPA), and combinations thereof, in an amount effective to reduce eicosanoid biosynthesis; and
a delivery formulation is selected from the group consisting of tablets, capsules, liquids, oils, suspensions, emulsions, solutions, and powders.

13. The composition as defined in claim 12, wherein reduce inflammation further comprises inhibiting eicosanoid biosynthesis.

14. The composition as defined in claim 13, wherein eicosanoid further comprises a compound selected from the group consisting of leukotriene, prostaglandin, and thromboxane.

15. A method for ameliorating inflammation-related conditions in humans and animals, the method comprising:
selecting a marine mollusk extract known to reduce inflammation;
selecting at least one fatty acid known to reduce inflammation;
incorporating an effective amount of marine mollusk extract and fatty acid into a suitable delivery formulation; and
administering the delivery formulation to humans and animals to ameliorate an inflammation-mediated disorder.

16. The method as defined in claim 15, wherein inflammation-mediated conditions may involve a disorder of at least one organ system selected from the group consisting of musculoskeletal, cardiovascular, neurological, respiratory, immunological, genito-urinary, endocrine, dermatological, renal, hepatic, and gastro-intestinal systems.

17. The method as defined in claim 15, wherein an inflammation-mediated condition is selected from the group consisting of osteoarthritis and rheumatoid arthritis.

18. The method as defined in claim 15, wherein the marine mollusk extract is derived from green-lipped mussel (Perna canaliculus).

19. The method as defined in claim 18, wherein the extract of green-lipped mussel is dried.

20. The method as defined in claim 18, wherein the extract of green-lipped mussel extract is in powder form.

21. The method as defined in claim 15, wherein the fatty acid is a polyunsaturated fatty acid.

22. The method as defined in claim 21, wherein the polyunsaturated fatty acid is selected from the group consisting of eicosapentaenoic acid (EPA), docosahexaenoic acid (DHA), eicosatetraenoic acid (ETA), docosapentaenoic acid (DPA), and combinations thereof.

23. The method as defined in claim 21, wherein the polyunsaturated fatty acid is derived from fish sources.

24. The method as defined in claim 15, wherein the marine mollusk extract inhibits eicosanoid biosynthesis.

25. The method as defined in claim 24, wherein eicosanoid further comprises a compound selected from the group consisting of leukotriene, prostaglandin, and thromboxane.

26. The method as defined in claim 15, the composition comprises a delivery formulation selected from the group consisting of tablets, capsules, liquids, oils, suspensions, emulsions, solutions, and powders.

27. A method for ameliorating inflammation-mediated conditions in humans and animals, the method comprising:
selecting a green-lipped mussel extract known to inhibit eicosanoid biosynthesis;
selecting at least one polyunsaturated fatty acid from the group consisting of eicosapentaenoic acid (EPA), docosahexaenoic acid (DHA), eicosatetraenoic acid (ETA), docosapentaenoic acid (DPA), and combinations thereof and known to inhibit eicosanoid biosynthesis;
incorporating an effective amount of green-lipped mussel extract and polyunsaturated fatty acid into a suitable delivery formulation; and
administering a delivery formulation to humans and animals.
